(19) United States
(12) Patent Application Publication
(10) Pub. No.: US 2023/0241325 A1
(43) Pub. Date: Aug. 3, 2023

(54) Medical Container, an Adaptor for Mounting Onto Said Medical Container, and a Drug Delivery Device Comprising Said Medical Container

(71) Applicant: Becton Dickinson France, Le Pont de Claix (FR)

(72) Inventor: Cédric Rivier, Voreppe (FR)

(21) Appl. No.: 17/920,232

(22) PCT Filed: Apr. 20, 2021
(86) PCT No.: PCT/EP2021/060154

§ 371 Date: Oct. 20, 2022

(30) Foreign Application Priority Data
Apr. 21, 2020 (EP) 20315203.8

(51) Int. Cl. A61M 5/32; A61M 39/10

(52) U.S. Cl. CPC A61M 5/32; A61M 39/10

(57) ABSTRACT

This medical container forms a reservoir for containing a medical product and has a longitudinal distal tip extending along a longitudinal axis. The distal tip defines a passageway in fluid communication with said reservoir. The distal tip includes a fitting portion configured to fit with a connector, and a mounting portion proximally located relative to said fitting portion and configured to slidably connect an adaptor to said distal tip. The mounting portion is configured to allow a free longitudinal movement of the adaptor relative to the distal tip when the adaptor is connected to the distal tip.

### CROSS-REFERENCE TO RELATED APPLICATIONS

This application is the United States national phase of International Application No. PCT/EP2021/060154 filed Apr. 20, 2021 and claims priority to European Patent Application No. 20315203.8 filed Apr. 21, 2020 the disclosures of which are hereby incorporated by reference in their entirety.

### BACKGROUND OF THE DISCLOSURE

### Field of the Disclosure

The present disclosure relates to a medical container, an adaptor for mounting onto said medical container, and a drug delivery device comprising said medical container.

### Description of Related Art

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction of injection, with respect to a medical container of the disclosure, and the “proximal direction” is to be understood as meaning the opposite direction to said direction of injection, that is to say the direction towards the user's hand holding a medical container as for an injection operation.

Basically, medical containers, such as for example syringes, may be made of a glass or plastic material. Preferably, the medical containers are made of glass for its high chemical passivity, its low gas permeability and high transparency, which allows an extended storage and an easy inspection.

The medical containers usually comprise a barrel forming a reservoir for containing a medical product. The barrel has a distal end in the form of a longitudinal tip defining an axial passageway through which the medical product is expelled from the container. However, this longitudinal tip does not allow parenteral administration by itself and must either comprise a staked needle or an adaptor allowing the connection of the syringe to a connector such as a needle hub or an intravenous (IV) line. This connector is typically screwed into the adaptor and engages the distal tip of the medical container to establish a fluid path between the reservoir and said connector.

It is known that the adaptor is strongly fixed to the longitudinal tip of the medical container in order to prevent accidental disengagement, either during connection of the connector onto the longitudinal tip, or caused by the fluid pressures within the drug delivery device and connector.

For example, the document WO2015/007650 discloses that adaptors may be secured around the longitudinal tip of the syringe by snap-fitting or friction force, for example by mechanical attaching means defined onto the longitudinal tip such as a groove or a ring.

The document WO2016/198580 alternatively discloses to provide adaptors with a mounting ring that is shaped and configured such that, when the adaptor is mounted around the longitudinal tip of the drug delivery device, a circumferential space is created between the mounting ring and the longitudinal tip, this circumferential space usually accommodating an adhesive layer in order efficiently to bond the adaptor to the longitudinal tip.

The document WO2010052517 further discloses an injection device and an adaptor allowing a safe connection of the adaptor on the injection device, in view of further connecting an IV (Intra Venous) connector to this injection device. The document EP2545956 discloses a drug delivery device having an end-piece and an adaptor for engaging around the end-piece so as to enable the safe connection of a connector on said end-piece. The document WO2012049532 discloses an adaptor intended to be used with a drug delivery device having an end-piece, the adaptor having a collar engageable around said-piece and enabling the safe connection of a connector on said collar.

It is however also important that the connector be properly screwed into the adaptor. Therefore, a user has to apply a sufficient torque when screwing the connector into the adaptor in order to get a proper fitting of the connector onto the distal tip of the medical container. Under-screwing or over-screwing of the connector into the adaptor may otherwise lead to a leakage or an ejection of the connector due to internal pressure when transferring a fluid between the container and the connector.

However, some connectors may abut against a distal face of the adaptor when being screwed into said adaptor. When such a contact happens, further rotation of the needle hub relative to the adaptor generates contact between the connector and the adaptor before the proper connection of the connector into the adaptor.

This early contact may give a user the false impression that a sufficient screwing torque is achieved, whereas the connector still does not properly fits onto the distal tip of the medical container. That is, if the user stops screwing, thinking that the connection is completed, the connector does not sufficiently engage the distal tip and a gap remains between the connector and an outer surface of this distal tip. Such a gap may lead to a leakage when transferring a fluid from the reservoir of the medical container to the connector.

There is therefore a need for a medical container preventing the risks of improper fitting and leakage between the connector and the distal tip even though the connector abuts against the distal face of the adaptor when being screwed into said adaptor.

### SUMMARY OF THE DISCLOSURE

An aspect of the disclosure is a medical container forming a reservoir for containing a medical product and having a longitudinal distal tip extending along a longitudinal axis A, the distal tip defining a passageway in fluid communication with said reservoir, wherein the distal tip includes
a fitting portion configured to fit with a connector; and
a mounting portion proximally located relative to said fitting portion and
configured to slidably connect an adaptor to said distal tip, characterized in that said mounting portion is configured to allow a free longitudinal movement of the adaptor relative to the distal tip when the adaptor is connected to the distal tip.

The medical container of the disclosure thus permits the adaptor to proximally slide along the distal tip if the connector abuts against a distal face of the adaptor. This prevents the screwing torque from increasing whereas the connector is not enough engaged onto the distal tip. Accordingly, a user will go on screwing the connector until this connector properly fits onto the distal tip of the medical container. As a result, risks of leakage are avoided.

In an embodiment, the mounting portion comprises guiding means configured to guide a translational movement of the adaptor relative to the distal tip and to prevent a rotation of the adaptor relative to the distal tip.

The translational movement is a longitudinal movement along the longitudinal axis A.

In an embodiment, the guiding means comprises at least one longitudinal bump, preferably three longitudinal bumps.

This allows a translational movement of the adaptor along the tip and blocks any rotation of the adaptor around said tip. The mounting portion of the distal tip, that may be made of a glass material or a plastic material, is indeed submitted to high stresses when the connector is secured to the adaptor. A single longitudinal bump is easier to manufacture. Three longitudinal bumps however provide an efficient guiding along the longitudinal direction A and an efficient blocking of any rotation around said longitudinal axis A.

In an embodiment, the mounting portion has a cylindrical shape.

The mounting portion thus guides the adaptor all along the mounting portion with reduced friction forces. The mounting portion has a constant outer diameter.

By cylindrical shape, it is meant that the mounting portion has the shape of any right cylinder, whose base may be a circle or a polygon, a square, an ellipse or any other non-circular geometrical shape. A mounting portion having a non-circular cross-section permits to guide the adaptor in the longitudinal direction while blocking a rotation of the adaptor around the distal tip. Such a mounting portion with a non-circular shape may form the guiding means in lieu of the at least one longitudinal bump. However, a mounting portion having a circular cross-section is easier to manufacture.

In an embodiment, the medical container comprises blocking means configured to limit the free longitudinal movement of the adaptor relative to the distal tip.

In an embodiment, the blocking means comprises a distal abutment surface and a proximal abutment surface.

The distal abutment surface prevents the removing of the adaptor from the distal tip of the medical container, and helps maintaining a closure cap of the medical container by blocking the adaptor in a distal-most position. The proximal abutment surface blocks the adaptor in a proximal-most position, thereby preventing the fact that the adaptor is too further engaged on the distal tip. In that case, it would not be possible anymore to engage the external thread of the connector into the inner thread of the adaptor.

In an embodiment, the distal abutment surface and the proximal abutment surface are respectively provided on a bump.

The adaptor is free to move relative to the distal tip along a predetermined amplitude a along the longitudinal axis A. By free longitudinal movement it is meant that the adaptor slides along the tip as soon as the adaptor is simply pushed proximally or distally in the axial direction. This predetermined amplitude a is comprised between 0.9 mm and 1.3 mm, preferably around 1.1 mm, along the longitudinal axis A. In other words, the blocking means may be configured so that the amplitude a of the adaptor free longitudinal movement is comprised between 0.9 mm and 1.3 mm, and is preferably around 1.1 mm. The greater the tip external diameter, the lower amplitude a needs to be. Conversely, the lower the tip external diameter, the greater amplitude a needs to be.

Another aspect of the disclosure is an adaptor for connecting the aforementioned medical container, said adaptor having connecting means configured to cooperate with complementary connecting means provided on a connector, and a mounting ring for mounting the adaptor onto the distal tip of the medical container, said mounting ring being configured to slidably engage the mounting portion of the distal tip.

In an embodiment, the mounting ring defines a through opening whose diameter is at least equal to or preferably greater than an outer diameter of the mounting portion of the distal tip.

There is thus no radial compression exerted by the mounting ring against the mounting portion of the distal tip.

Preferably, the difference between the diameter of the through opening and the outer diameter of the mounting portion is comprised between 0.1 mm and 0.3 mm, typically 0.2 mm.

In an embodiment, the mounting ring is in the form of a frustoconical disc.

The disc is distally inclined relative to a tubular wall of the adaptor. This reduces the insertion force while increasing the adaptor pull out force.

Another aspect of the disclosure is a drug delivery device comprising the aforementioned medical container and the aforementioned adaptor, wherein the adaptor is slidably mounted onto the mounting portion of the distal tip.

The adaptor is free to move relative to the distal tip along a predetermined amplitude, said predetermined amplitude being preferably comprised between 0.9 mm and 1.3 mm along the longitudinal axis A.

In an embodiment, the drug delivery device further includes a needle hub having an inner conduit configured to fit onto the distal tip and outer wings configured to engage an inner thread of the adaptor, said needle hub including a ramp for guiding a user's finger towards a pivoting arm in order to move the pivoting arm back towards a safety position wherein the pivoting arms covers a needle.

### BRIEF DESCRIPTION OF THE DRAWINGS

The disclosure and the advantages arising therefrom will clearly emerge from the detailed description that is given below with reference to the appended drawings as follows:

FIG. 1 is a perspective view of a medical container according to an embodiment of the disclosure,

FIG. 2 is a perspective view of an adaptor according to an embodiment of the disclosure,

FIG. 3 is a cross-section view of a drug delivery device according to an embodiment of the disclosure,

FIGS. 4A and 4B are cross-section views of drug delivery devices according to an embodiment of the disclosure, when a needle hub is screwed into the adaptor,

FIG. 5 is a perspective view of a needle hub for cooperating with a drug delivery device according to an embodiment of the disclosure,

FIG. 6 is a cross section view of a needle hub for cooperating with a drug delivery device according to an embodiment of the disclosure.

### DETAILED DESCRIPTION

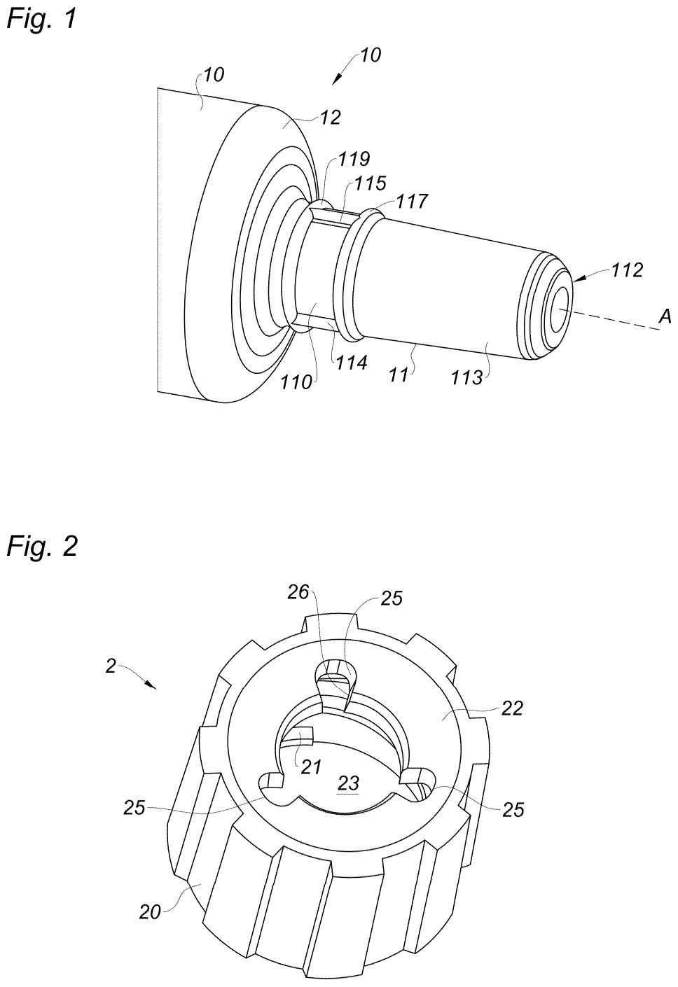

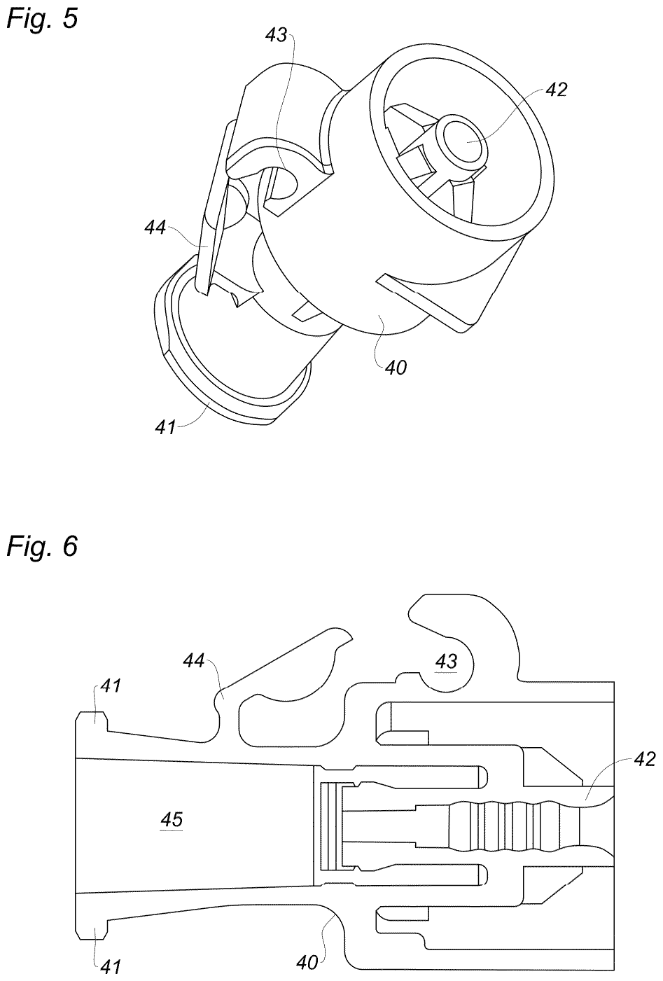

With reference to FIGS. 1 to 4B is shown a medical container 1, an adaptor 2 and a drug delivery device 3 according to an embodiment of the disclosure. The drug delivery device 3 is intended to cooperate with a connector such as a needle hub 40 as shown on FIGS. 5 and 6.

As illustrated on FIG. 1, the medical container 1 comprises a barrel 10 defining a reservoir for containing a medical product. A distal tip 11 defining a passageway in fluid communication with the reservoir longitudinally extends along a longitudinal axis A of the medical container 1. The medical container 1 may be made of a glass material or a plastic material. The distal tip 11 may consequently be made either by glass forming or by injection molding.

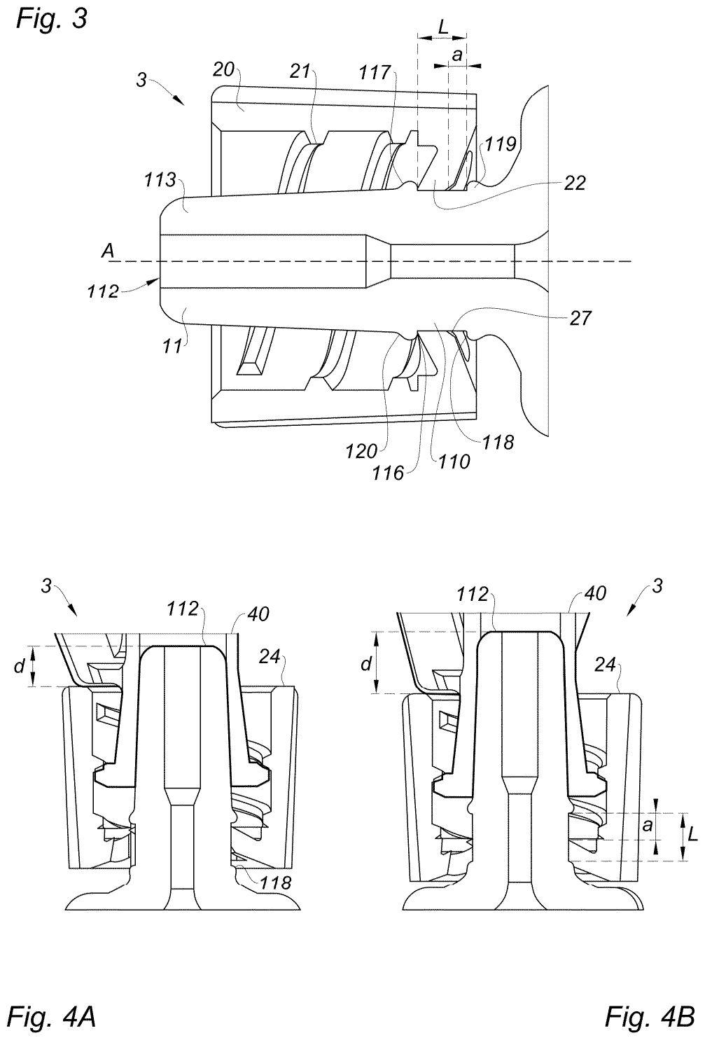

The distal tip 11 of the medical container 1 has a mounting portion 110. The mounting portion 110 is configured to receive a mounting ring 22 of the adaptor 2 for connecting the adaptor 2 onto the tip 11, as illustrated on FIG. 3. The mounting portion 110 preferably extends in a proximal region of the distal tip 11. More specifically, the mounting portion 110 may be closer to a distal shoulder 12 of the barrel 10 than a distal face 112 of the longitudinal tip 11. Typically, the mounting portion 110 is disposed in the proximal-most third of the distal tip 11 if said distal tip 11 is divided into three equal thirds.

The distal tip 11 further comprises a fitting portion 113. The fitting portion 113 distally extends from the mounting portion 110. Said fitting portion 113 is configured to fit with an inner conduit of the needle hub 40 so as to establish a fluid path from the reservoir of the medical container 1 to a needle (not shown) of the needle hub 40. The fitting portion 113 preferably has a frustoconical shape.

According to the disclosure, the mounting portion 110 of the distal tip 11 comprises guiding means for guiding a longitudinal sliding movement of the adaptor 2 along the longitudinal axis A and for blocking a rotational movement of the adaptor 2 relative to the distal tip 11 around said longitudinal axis A.

As a result, the adaptor 2 is free to move relative to the distal tip 11 along a predetermined amplitude a. The predetermined amplitude a is defined by the difference between the length L of the mounting portion 110 and the thickness of the mounting ring 22, as illustrated in FIG. 3. This predetermined amplitude a may be comprised between 0.9 mm and 1.3 mm, preferably around 1.1 mm, along the longitudinal axis A. The adaptor 2 is free to slide along the longitudinal axis A within the predetermined amplitude a, i.e. between the distal-most position and the proximal-most position of the adaptor 2 when said adaptor 2 is mounted onto the distal tip 11.

This longitudinal movement of the adaptor 2 relative to the tip 11 permits to vary the distance d between the distal face 112 of the tip 11 and a distal face 24 of the adaptor 2 as visible on FIGS. 4A and 4B. As a result, the adaptor 2 may be pushed in a proximal direction, thereby increasing the distance d, by the needle hub 40 abutting against a distal face 24 of the adaptor 2. This allows the distal tip 11 to properly fit into an inner conduit of the needle hub 40. Risks of leakage due to an insufficient screwing of the needle hub 40 into the adaptor 2 because of a contact between the needle hub 40 and the distal face 24 of the adaptor 2 are thus avoided.

With reference to FIG. 1, the guiding means may comprise at least one longitudinal bump 114. The at least one longitudinal bump 114 extends in a straight direction parallel to the longitudinal axis A. Having a bump, that protrudes from an outer wall of the mounting portion 110, instead of a longitudinal groove that would form a recess with regard to said outer wall, has the advantage of strengthening, not weakening, said mounting portion 110 and said tip 11.

It should be noted that the outer diameter of the mounting portion 110, without bumps 114, 117, 119, is equal to or lower than, preferably equal to, the greatest outer diameter of the fitting portion 113 of the distal tip 11. In other words, the mounting portion 110 of the distal tip 11 is preferably no thinner than the rest of the tip 11. As a result, the mounting portion 110 is devoid of any groove or recess. This limits the risk of breakage, especially when the medical container 1 is made of glass.

As shown on FIG. 1, the at least one longitudinal bump 114 may be in the form of an longitudinal rib extending all along the mounting portion 110.

The at least one longitudinal bump 114 may have a rounded shape, as illustrated on FIG. 1. The at least one bump 114 may define recessed lateral edges 115.

Preferably, the guiding means may comprise three longitudinal bumps 114. Having three longitudinal bumps 114 offers a satisfactory repartition of the screwing torque around the tip 11 in order to avoid breakage without complicating the manufacturing process, especially when the tip 11 is made of glass. The three longitudinal bumps 114 may be regularly distributed around the mounting portion 110. The guiding means may however have less or more than three longitudinal bumps 114.

The mounting portion 110 preferably has cylindrical shape. Alternatively or complementarily to the at least one longitudinal bump 114, the guiding means may comprise the mounting portion 110 having a square, oval, polygonal or any other non-circular cross-section shape. Such a shape permits the adaptor 2 to slide along the mounting portion 110 without rotating around said mounting portion 110.

With reference to FIGS. 1 or 3 to 4B, the mounting portion 110 may further comprise blocking means for blocking the translational movement of the adaptor 2 relative to the tip 11.

The blocking means may comprise a first abutment surface 116 for blocking the adaptor 2 respectively in the distal direction. The first abutment surface 116 further prevents the pull out of the adaptor 2. In a distal-most position (FIG. 3), the mounting ring 22 abuts against the first abutment surface 116.

The first abutment surface 116 defines the distal end of the mounting portion 110. The first abutment surface 116 may be arranged at the proximal side of a first orthoradial bump 117. This first orthoradial bump 117 protrudes from an outer wall of the distal tip 11. Having a first abutment surface 116, that is defined by a bump 117 instead of being defined by a shoulder. The first orthoradial bump 117 may be in the form of a circumferential rib extending all around the tip 11.

With reference to FIG. 3, the first orthoradial bump 117 may have a ramp portion 120 provided on a distal side of said bump 117 in order to ease the passage of the mounting ring 22 of the adaptor 2 over said first orthorodial bump 117 in the proximal direction. This helps the user mount the adaptor 2 onto the mounting portion 110 of the tip 11.

The blocking means may comprise a second abutment surface 118 for blocking the adaptor 2 respectively in the proximal direction. In a proximal-most position (FIG. 4B), the mounting ring 22 abuts against the second abutment surface 118.

The second abutment surface 118 defines the proximal end of the mounting portion 110. The second abutment surface 118 may be arranged at the distal side of a second orthoradial bump 119. This second orthoradial bump 119 protrudes from an outer wall of the distal tip 11. Having a second abutment surface 118, that is defined by a bump 119 instead of being defined by a shoulder. The second orthoradial bump 119 may be in the form of a circumferential rib extending all around the tip 11.

The second abutment surface 118 may be located such that the distance d between the distal face of the tip 11 and the distal face 24 of the adaptor 2, when said adaptor 2 is connected to the tip 11, is no more than 2.7 mm. This allows the needle hub 40 to engage the adaptor 2, even though the adaptor 2 is in the proximal-most position. Indeed, if the distance d becomes too high, the needle hub 40 would otherwise not be able to engage the adaptor 2. More specifically, the maximum distance d of 2.7 mm allows the thread of the needle hub 40 to engage the thread 21 of the adaptor 2 when the distal tip 11 has a high external diameter.

The blocking means may define the length L of the mounting portion 110. With reference to FIG. 3, this length L may be defined by the distance between the first and the second abutment surfaces 116, 118.

FIG. 4A shows a needle hub 40 that is fitted to a distal tip 11 having a first diameter whereas FIG. 4B shows a needle hub 40 that is fitted to a distal tip 11 having a second diameter that is smaller than said first diameter. As can be seen on these Figs., the sliding movement of the adaptor 2 along the distal tip 11 permits that the needle hub 40 reaches a predetermined position relative to the distal tip 11 wherein said needle hub 40 properly fits onto the tip 11. This predetermined position is advantageously defined as being the leakage limit of the needle hub 40 as defined in the ISO 80369-7 (2016) (paragraphs 6.1 and 6.2). Whether the needle hub 40 is under connected and has not reach said position, a leakage may occur. The sliding movement allows the needle hub 40 to reach the leakage limit position despite the contact that may occur between the needle and the adaptor 2 distal face.

With reference to FIGS. 2 and 3, the disclosure also relates to an adaptor 2 configured to be connected to the mounting portion 110 of the tip 11.

The adaptor 2 has a tubular wall 20 defining an inner cavity for receiving the needle hub 40. The inner cavity is provided with connecting means, such as an inner thread 21, for securing the needle hub 40 to the adaptor 2.

The adaptor 2 further has a mounting ring 22 inwardly protruding from a proximal end of the adaptor 2. The mounting ring 22 allows connecting the adaptor 2 onto the distal tip 11.

The mounting ring 22 defines a through opening 23 for receiving the mounting portion 110 of the tip 11. The through opening 23 may be shaped to complementarily engage the mounting portion 110 of the tip 11. The thickness of the mounting ring 22 should be high enough to prevent the adaptor 2 pull out, but needs to be low enough to avoid increasing the length of the mounting portion 110 and thus the length of the distal tip 11. For instance, said thickness may be approximately comprised between 0.6 mm and 1.4 mm.

The width or diameter of said through opening 23 may be at least equal to, preferably greater than, the outer width or diameter of the mounting portion 110. As a result, a clearance is provided between the mounting ring 22 and the mounting portion 110. This favors the sliding movement of the adaptor 2 relative to the tip 11.

With reference to FIG. 2, the mounting ring 22 comprises at least one guiding window 25. The at least one window 25 is configured to engage the at least one longitudinal bump 114 in order to guide the sliding movement of the adaptor 2 relative to the tip 11 and to block any rotation of the adaptor 2 relative to said tip 11. To that end, the at least one guiding window 25 and the longitudinal bump 114 may be complementarily shaped, while allowing the free sliding movement of the adaptor 2 relative to the distal tip 11. Preferably, the at least one guiding window 25 and the at least one longitudinal bump 114 are configured such that the adaptor 2 and the distal tip 11 are snap-fitted. For example, the at least one guiding window 25 may be slightly larger than the at least one longitudinal bump 114. The adaptor 2 may comprise as many guiding windows 25 as longitudinal bumps 114, for example three as illustrated on FIG. 2.

As shown on FIG. 2, the at least one guiding window 25 may open in the through opening 23 defined by said mounting ring 22. The adaptor 2 may thus be clipped onto the mounting ring 22 and the at least one longitudinal bump 114. The at least one window 25 may have lateral edges 26 configured to engage the corresponding lateral edges 115 of the at least one longitudinal bump 114. This improves the clipping of the mounting ring 22 onto the at least one longitudinal bump 114. The lateral edges 26 may be part of snap-fitting means for connecting the adaptor 2 to the mounting portion 110.

With reference to FIG. 3, the mounting ring 22 of the adaptor 2 may be in the form of a resilient frustoconical disc. The frustoconical disc is inclined relative to the longitudinal axis A and points towards the distal direction. This increases the adaptor 2 pull out force. This further helps the passage of the adaptor 2 over the first orthoradial bump 117. The inclined proximal side of the mounting ring 22 cooperates with the ramp portion 120 of said first orthoradial bump 117 to permit resilient deformation of the mounting ring 22. A beveled edge 27 may further be provided at the proximal side of the mounting ring 22.

The adaptor 2 may be made of any rigid polymer adapted to medical use, such as high density polyethylene (PE), polypropylene (PP), polycarbonate (PC), acrylonitrile butadiene styrene (ABS), polyoxymethylene (POM), polystyrene (PS), polybutylene terephthalate (PBT), polyamide (PA), and combinations thereof. To simplify its manufacturing, the adaptor 2 preferably consists of a single piece of material, preferably of a light-transmitting material. The adaptor 2 may be made by injection molding.

With reference to FIGS. 5 and 6, the disclosure further relates to a drug delivery device 3 comprising said medical container 1 and said adaptor 2 connected to the distal tip 11 of the medical container 1. The drug delivery device 3 may be a syringe, such as prefilled or pre-fillable syringe.

The drug delivery device 3 may comprise a connector such as a needle hub 40 as shown on FIGS. 5 and 6. The needle hub 40 comprises a proximal end defining an inner conduit 45 for receiving the distal tip 11 of the medical container 1. The distal end is provided with connecting means, such as two diametrically opposite outer wings 41, configured to engage the connecting means of the adaptor 2, such as the inner thread 21, in order to secure the needle hub 40 to the adaptor 2. The needle hub 40 further includes a distal end that may be provided with a mounting port 42 for mounting an injection needle and thereby establish a fluid path from the reservoir to the injection needle. A pivoting arm (not shown) may be connected at a pivot connection 43 in the form of a hook, said pivoting arm being configured to cover or unveil the injection needle. In order to help the user move the pivoting arm back towards the needle, the needle hub 40 may comprise a ramp 44 configured to guide a user's finger against the pivoting arm. For example, the needle hub 40 may be a BD Eclipse®.

The disclosure thus permits a longitudinal movement of the adaptor 2 relative to the tip 11 of the medical container 1. This axial freedom allows a proper fitting between the needle hub 40 and the distal tip 11 of the medical container 1 in any situation. More specifically, the longitudinal movement of the adaptor 2 relative to the tip 11 permits the needle hub 40 to reach the leakage limit position even when the needle hub 40 abuts against the distal face of adaptor 2. The longitudinal movement of the adaptor 2 relative to the tip 11 permits the needle hub 40 to reach the leakage limit position regardless of the distal tip 11 diameter. As a result, risks of leakage due to unproper fitting between the tip 11 and the needle hub 40 are avoided.

1. A medical container forming a reservoir for containing a medical product and having a longitudinal distal tip extending along a longitudinal axis (A), the distal tip defining a passageway in fluid communication with said reservoir, wherein the distal tip comprises
a fitting portion configured to fit with a connector; and
a mounting portion proximally located relative to said fitting portion and configured to slidably connect an adaptor to said distal tip,
wherein said mounting portion is configured to allow a free longitudinal movement of the adaptor relative to the distal tip when the adaptor is connected to the distal tip,
wherein the mounting portion comprises guiding means configured to guide a translational movement of the adaptor relative to the distal tip and to prevent a rotation of the adaptor relative to the distal tip,
the guiding means comprising at least one longitudinal bump, preferably three longitudinal bumps, configured to engage at least one complementarily shaped guiding window of a mounting ring of the adaptor, and
the least one longitudinal bump has a rounded shape and defines recessed lateral edges configured to receive lateral edges of said at least one guiding window, thereby allowing a snap-fit connection between the adaptor and the mounting portion of the distal tip.

2. The medical container according to claim 1, wherein the mounting portion has a cylindrical shape.

3. The medical container according to claim 1, comprising blocking means configured to limit the free longitudinal movement of the adaptor relative to the distal tip.

4. The medical container according to claim 3, wherein the blocking means comprises a distal abutment surface and a proximal abutment surface.

5. The medical container according to claim 4, wherein the distal abutment surface and the proximal abutment surface are respectively provided on a bump.

6. The medical container according to claim 3, wherein the blocking means are configured so that an amplitude of the adaptor free longitudinal movement is comprised between 0.9 mm and 1.3 mm, and is preferably around 1.1 mm.

7. An adaptor for connecting the medical container according to claim 1, said adaptor having connecting means configured to cooperate with complementary connecting means provided on a connector, and a mounting ring for mounting the adaptor onto the distal tip of the medical container, said mounting ring being configured to slidably engage the mounting portion of the distal tip, wherein the mounting ring comprises at least one guiding window configured to engage an at least one complementarily shaped longitudinal bump of said mounting portion in order to guide the sliding movement of the adaptor relative to the tip and to block any rotation of the adaptor relative to said tip, and wherein this at least one window has lateral edges configured to engage corresponding lateral edges of the at least one longitudinal bump having a rounded shape, said lateral edges forming snap-fitting means for connecting the adaptor to the mounting portion.

8. The adaptor according to claim 7, wherein the mounting ring defines a through opening whose diameter is at least equal to or preferably greater than an outer diameter of the mounting portion of the distal tip.

9. The adaptor according to claim 7, wherein the mounting ring is in the form of a frustoconical disc.

10. A drug delivery device comprising the medical container according to claim 1 and the adaptor according to any of claims 7-9, wherein the adaptor is slidably mounted onto the mounting portion of the distal tip.

11. The drug delivery device according to claim 10, wherein the drug delivery device further includes a needle hub having an inner conduit configured to fit onto the distal tip and outer wings configured to engage an inner thread of the adaptor, said needle hub including a ramp for guiding a user's finger towards a pivoting arm in order to move the pivoting arm back towards a safety position wherein the pivoting arms covers a needle.
